(19) United States
(12) Patent Application Publication
(10) Pub. No.: US 2026/0263496 A1
(43) Pub. Date: Sep. 10, 2026

(54) TOPICAL APPLICATION OF CLITOCINE FOR THE TREATMENT OF PREMATURE STOP CODON RELATED DISEASES

(71) Applicant: Verein zur Förderung der Universitätsklinik für Dermatologie und Allergologie Salzburg, Salzburg (AT)

(72) Inventors: Johann BAUER, Salzburg (AT); Roland WAGNER, Salzburg (AT); Johanna ZANDANELL, Freiburg im Breisgau (DE); Michael WIESSNER, Salzburg (AT)

(21) Appl. No.: 19/073,197

(22) Filed: Mar. 7, 2025

(51) Int. Cl. A61K 31/7064; A61K 9/00; A61P 17/00

(52) U.S. Cl. CPC A61K 31/7064; A61K 9/0014; A61P 17/00

(57) ABSTRACT

The present invention relates to a method for treating a skin disease responsive to modulation of premature translation termination, comprising administering to a mammalian patient in need thereof an effective amount of clitocine as a composition for topical use comprising the compound and a pharmaceutically acceptable carrier or diluent. The topical treatment is surprisingly effective and has less side effects than known treatments. The present invention further relates to a method for modulating premature translation termination in a mammalian skin cell, comprising contacting a mammalian skin cell exhibiting premature translation termination with an effective amount of clitocine as a composition for topical use comprising the compound and a pharmaceutically acceptable carrier or diluent.

### FIELD OF THE INVENTION

The present invention relates to a method for treating a skin disease responsive to modulation of premature translation termination, comprising administering to a mammalian patient in need thereof an effective amount of clitocine as a composition for topical use comprising the compound and a pharmaceutically acceptable carrier or diluent. The topical treatment is surprisingly effective and has less side effects than known treatments. The present invention further relates to a method for modulating premature translation termination in a mammalian skin cell, comprising contacting a mammalian skin cell exhibiting premature translation termination with an effective amount of clitocine as a composition for topical use comprising the compound and a pharmaceutically acceptable carrier or diluent.

### BACKGROUND OF THE INVENTION

Epidermolysis bullosa (EB) comprises a very heterogeneous group of inherited genodermatoses. In response to minor mechanical trauma, blisters, peeling, erosions or ulcerations of varying degrees are characteristic of this disease. Largely, morbidity and mortality depend on extracutaneous manifestations, making EB a multi-system disease. EB is considered a rare or orphan disease.

Structural proteins of the dermo-epidermal junction are responsible for functional integrity of the skin and mucous membranes and are key to understanding the pathophysiology of EB. The genes coding for these proteins are also expressed in epithelialized and non-epithelialized tissues, explaining the various extracutaneous manifestations in the musculoskeletal system, the respiratory, gastrointestinal and urogenital tract. Currently, mutations in 16 different genes are known to cause EB (Bardhan A, Bruckner-Tuderman L, Chapple I L C, et al. Epidermolysis bullosa. Nat Rev Dis Primers. 2020; 6 (1): 78. doi: 10.1038/s41572-020-0210-0, Has C, Bauer μW, Bodemer C, et al. Consensus reclassification of inherited epidermolysis bullosa and other disorders with skin fragility. Br J Dermatol. 2020; 183 (4): 614-627. doi: 10.1111/bjd.18921). The mutations may cause a gradual loss of function, reduction or complete absence of the corresponding protein, leading to a significant genotype/phenotype diversity.

Based on the level of cleavage within the skin, hereditary EB is classified into four major types. Blistering within the epidermis defines EB simplex. Predominantly, the defects are caused by mutations in keratins 5 and 14 and plectin (KRT5, KRT14, PLEC). Junctional EB is characterized by blister formation within the lamina lucida due to defects in genes coding for laminin 332 (LAMA3, LAMB3, LAMC2), type XVII collagen (COL17A1), integrin α6β4 (ITGA6, ITGB4) or integrin α3 subunit (ITGA3). Skin cleavage beneath the lamina densa, presented in dystrophic EB, is caused by mutations in the gene coding for type VII collagen (COL7A1). Lastly, Kindler EB, classified by various patterns of tissue defects, is associated with mutations in the gene coding for kindlin 1 (KIND1, also FERMT1).

Premature termination codon (PTC) readthrough or nonsense suppression therapies represent an intriguing avenue of EB research. Nonsense mutations—accounting for the most severe subtypes of EB (Welponer T, Prodinger C, Pinon-Hofbauer J, et al. Clinical perspectives of gene-targeted therapies for epidermolysis bullosa. Dermatol Ther. 2021; 11 (4): 1175-1197. doi: 10.1007/s13555-021-00561-5)—are single base-pair mutations that introduce a premature termination codon (PTC) within the coding region of the affected gene. When the ribosome encounters a PTC, translation is halted and terminated prematurely. As a result, a truncated and non-functional protein is released. PTC therapeutics interfere with translation by reducing the ribosome's fidelity, which consequently reads pass a PTC and continues synthesizing a mature protein.

Numerous studies reported the value of SCR therapy for various diseases triggered by PTC mutations (Lee H L, Dougherty J P. Pharmaceutical therapies to recode nonsense mutations in inherited diseases. Pharmacol Ther. 2012; 136 (2): 227-266. doi: 10.1016/j.pharmthera. 2012.07.007). Stop codon readthrough (SCR)-inducing compounds can be classified as follows: aminoglycoside antibiotics, non-aminoglycoside antibiotics and non-aminoglycoside compounds. Rather than binding the ribosomal decoding centre, some of these compounds act by interfering with other layers of the translation process. Unfortunately, long-term administration of aminoglycosides harbours the risk of significant side effects, including ototoxicity and nephrotoxicity.

Several non-aminoglycoside antibiotics (e.g., erythromycin) can induce PTR readthrough. Although infrequent, serious side effects include hepatotoxicity and cardiac arrhythmia.

Finally, non-aminoglycoside compounds (e.g., PTC-124/ataluren, amlexanox, clitocine, artesunate and atazanavir), operating independently or acting synergistically with aminoglycosides, may hold therapeutic potential for SCR induction. PTC-124 (ataluren). However, controversial results have questioned the drug's effect on SCR and the development of the drug might have been influenced by the choice of assay implemented during the drug discovery process.

Welponer et al. further describe that clitocine ((2R,3R,4S,5R)-2-[(6-Amino-5-nitropyrimidin-4-yl)amino]-5-(hydroxymethyl)oxolane-3,4-diol or 6-Amino-5-nitro-4-(β-D-ribo-furanosylamino)pyrimidine) is a nucleoside analogue isolated from mushrooms that substitutes for the adenosines of PTCs during transcription and thus directly suppresses nonsense mutations. Clitocine is a potent and efficacious stimulator of SCR as demonstrated by restoration of functional p53 protein in p53 nonsense-mutated cells in vitro and inhibition of tumour growth in a xenograft model in vivo. In the latter, mice were subcutaneously injected with concentrations of 0.3 or 3.0 mg/kg five times per week or 20 mg/kg once per week and compared to a control group. Thus, clitocine was said to represent a promising treatment opportunity for cancers caused by nonsense mutations within tumour suppressor genes. Tolerability and side effects, though, were not investigated in this study (see Friesen W J, Trotta C R, Tomizawa Y, et al. The nucleoside analog clitocine is a potent and efficacious readthrough agent. RNA. 2017; 23 (4): 567-577. doi: 10.1261/rna.060236.116). Clitocine had not been involved in any clinical trial to date, and besides, not yet been mentioned in the context of EB. However, initial studies demonstrate high efficacy in suppressing the PTC originating from the LAMB3 hotspot mutation R635X (NM_000228.3:c.1903C>T; NP_000219.2:p.Arg635Ter) even at concentrations in the sub-micromolar range. Consequently, high levels of R635X SCR were achieved, as indicated by prominent firefly luciferase activity in a specialized reporter assay. Specifically, treatment with G418 (0.5 mg/mL) resulted in a normalized fold induction of 10 compared to DMSO control. At the same time, we recorded a normalized fold induction of 30 upon treatment with clitocine (1 μM).

Clitocine is a fungal nucleoside isolate with anticancer activity in vitro. The drug has the following formula.

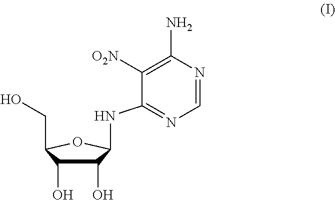

The total synthesis of clitocine has been reported. (Moss et al., J. Med. Chem., 31:786-790 (1988) and Kamikawa et al., J. Chem. Soc. Chem. Commun. 195 (1988)). Clitocine has been reported to possess insecticidal activity and cytostatic activity against leukemia cell lines (Kubo et al., Tet. Lett 27:4277 (1986) and Moss et al., J. Med. Chem. 31:786-790 (1988)).

Clitocine is also known as read-through agent. Clitocine seems to be developed by PTC Therapeutics (NJ, USA). Friesen W J, et al. (in: The nucleoside analog clitocine is a potent and efficacious readthrough agent. RNA. 2017 April; 23 (4): 567-577. doi: 10.1261/rna.060236.116. Epub 2017 Jan. 17. PMID: 28096517; PMCID: PMC5340919) describe that nonsense mutations resulting in a premature stop codon in an open reading frame occur in critical tumor suppressor genes in a large number of the most common forms of cancers and are known to cause or contribute to the progression of disease. Low molecular weight compounds that induce readthrough of nonsense mutations offer a new means of treating patients with genetic disorders or cancers resulting from nonsense mutations. They have identified the nucleoside analog clitocine as a potent and efficacious suppressor of nonsense mutations, and, determined that incorporation of clitocine into RNA during transcription is a prerequisite for its readthrough activity; the presence of clitocine in the third position of a premature stop codon directly induces readthrough. They demonstrate that clitocine can induce the production of p53 protein in cells harboring p53 nonsense-mutated alleles. In these cells, clitocine restored production of full-length and functional p53 as evidenced by induced transcriptional activation of downstream p53 target genes, progression of cells into apoptosis, and impeded growth of nonsense-containing human ovarian cancer tumors in xenograft tumor models. Thus, clitocine allegedly induces readthrough of nonsense mutations by a previously undescribed mechanism and represents a novel therapeutic modality to treat cancers and genetic diseases caused by nonsense mutations.

WO 2004/009610A2 describes nucleoside compounds, compositions comprising the compounds and methods for treating or preventing diseases associated with nonsense mutations of mRNA by administering these compounds or compositions.

U.S. Pat. No. 7,449,570B2 relates to a method of modulating premature translation termination and/or nonsense-mediated mRNA decay in a cell, comprising contacting a cell exhibiting premature translation termination and/or nonsense-mediated mRNA decay with an effective amount of a compound, which seems to include clitocine. Pharmaceutical compositions and single unit dosage forms comprising a compound of the invention, or a pharmaceutically acceptable polymorph, prodrug, salt, solvate, hydrate, or clathrate thereof are also described.

U.S. Pat. No. 7,291,603B2 relates to a method of treating a disease resulting from a somatic nonsense mutation in the p53 gene comprising administering to a patient in need thereof an effective amount of a compound, which seems to include clitocine. Transdermal and topical dosage forms are disclosed and include creams, lotions, ointments, gels, solutions, emulsions, suspensions, or other forms known to one of skill in the art.”

WO 2012/174126A1 discloses methods for treating a NAD comprising administering to a patient suffering from a NAD an inhibitor of NMD and a nonsense suppressor, whereby degradation of NMD susceptible mRNA is decreased and translation termination at a misplaced nonsense codon is blocked. The nonsense suppressor may be clitocine.

Zandanell J et al. (in: Stop codon readthrough as a treatment option for epidermolysis bullosa—Where we are and where we are going. Exp Dermatol. 2024 March; 33 (3): e15042. doi: 10.1111/exd.15042. PMID: 38459626) discloses that in the context of rare genetic diseases caused by nonsense mutations, the concept of induced stop codon readthrough (SCR) represents an attractive avenue in the ongoing search for improved treatment options. Epidermolysis bullosa (EB)-exemplary for this group of diseases-describes a diverse group of rare, blistering genodermatoses. Characterized by extreme skin fragility upon minor mechanical trauma, the most severe forms often result from nonsense mutations that lead to premature translation termination and loss of function of essential proteins at the dermo-epidermal junction. Since no curative interventions are currently available, medical care is mainly limited to alleviating symptoms and preventing complications. Complementary to attempts of gene, cell and protein therapy in EB, SCR represents a promising medical alternative. While gentamicin has already been examined in several clinical trials involving EB, other potent SCR inducers, such as ataluren, may also show promise in treating the hitherto non-curative disease. In addition to the extensively studied aminoglycosides and their derivatives, several other substance classes—non-aminoglycoside antibiotics and non-aminoglycoside compounds—are currently under investigation. The extensive data gathered in numerous in vitro experiments and the perspectives they reveal in the clinical setting will be discussed in this review.

Accordingly, small molecule therapeutics or prophylactics that suppress premature translation termination by mediating the misreading of the nonsense codon would be useful for the treatment of a number of diseases, in particular diseases of the skin, such as genodermatoses. Other objects and advantages will readily become apparent for the person of skill from studying the following more detailed description and examples.

### BRIEF SUMMARY

The present invention is based in part on the discovery of small molecules that modulate premature translation termination. The present invention encompasses methods for the topical use of clitocine.

The object of the present invention is solved by providing a method of treating a skin disease responsive to modulation of premature translation termination, comprising administering to a mammalian patient in need thereof an effective amount of a compound having the structure of formula I:

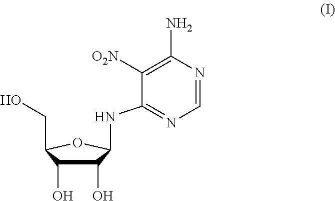

or a pharmaceutically acceptable salt, hydrate, solvate, clathrate, racemate or stereoisomer thereof, wherein the compound, or a pharmaceutically acceptable salt, hydrate, solvate, clathrate or stereoisomer thereof, is administered as a composition for topical use comprising the compound and a pharmaceutically acceptable carrier or diluent.

Preferred is the method according to the present invention, wherein the skin disease is selected from a genodermatosis, such as epidermolysis bullosa.

Further preferred is the method according to the present invention, wherein the premature translation termination is caused by mutation R635X in the gene LAMB3, or a mutation selected from Arg2332X, Arg2541X, Gly2599X, and Arg2685X in the gene COL7A1.

In another aspect, the object of the present invention is solved by a method of modulating premature translation termination in a mammalian skin cell, comprising contacting a mammalian skin cell exhibiting premature translation termination with an effective amount of a compound having the structure of formula I:

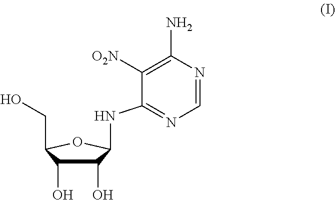

or a pharmaceutically acceptable salt, hydrate, solvate, clathrate, racemate or stereoisomer thereof, wherein the compound, or a pharmaceutically acceptable salt, hydrate, solvate, clathrate or stereoisomer thereof, is administered to the skin cell as a composition for topical use comprising the compound and a pharmaceutically acceptable carrier or diluent.

Preferred is the method according to the present invention, wherein the premature translation termination is caused by mutation R635X in the gene LAMB3, or a mutation selected from Arg2332X, Arg2541X, Gly2599X, and Arg2685X in the gene COL7A1.

Further preferred is the method according to the present invention, wherein the cell is a patient-derived cell or a cell line, in particular a keratinocyte, such as a genetically modified keratinocyte.

### DETAILED DESCRIPTION

The present invention is based in part on the discovery of small molecules that modulate premature translation termination and/or nonsense-mediated mRNA decay. The present invention encompasses a method of treating a skin disease responsive to modulation of premature translation termination, comprising administering to a mammalian patient in need thereof an effective amount of a compound having the structure of formula I:

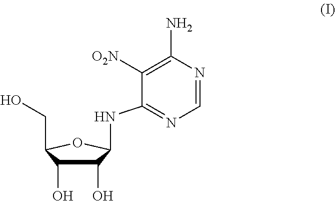

or a pharmaceutically acceptable salt, hydrate, solvate, clathrate, racemate or stereoisomer thereof, wherein the compound, or a pharmaceutically acceptable salt, hydrate, solvate, clathrate or stereoisomer thereof, is administered as a composition for topical use comprising the compound and a pharmaceutically acceptable carrier or diluent.

Without being limited by theory, the invention encompasses methods for modulating premature translation termination in a cell, including mammalian cells in a living host. The invention further encompasses a method for suppressing premature translation termination in a cell comprising contacting a cell exhibiting premature translation termination with an effective amount of a compound of formula (I) administered as a composition for topical use comprising the compound and a pharmaceutically acceptable carrier or diluent.

It was found that the compound according to formula (I) is particularly effective for modulating premature translation termination in a cell, in particular when administered topically. The compound provides a higher translation and has substantially less side-effects than gentamycin (see also examples below).

As used herein, the term “pharmaceutically acceptable salts” refer to salts prepared from pharmaceutically acceptable non-toxic acids or bases including inorganic acids and bases and organic acids and bases. Suitable pharmaceutically acceptable base addition salts for the compound of formula I include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “polymorph” refers to solid crystalline forms of a compound of formula I or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g. differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g. tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of formula I or a salt thereof, that further includes a stoichiometric or non-stoichiometric amount of water bound by non-covalent intermolecular forces.

As used herein, the term “clathrate” means a compound of formula I or a salt thereof in the form of a crystal lattice that contains spaces (e.g. channels) that have a guest molecule (e.g., a solvent or water) trapped within.

Preferred is the method according to the present invention, wherein the composition for topical use is selected from a pharmaceutical composition comprising transdermal or topical dosage forms, such as, for example creams, lotions, ointments, gels, solutions, emulsions, suspensions, and combinations thereof. Pharmaceutical compositions and single unit dosage forms comprising a compound of formula I, or a pharmaceutically acceptable polymorph, prodrug, salt, solvate, hydrate, or clathrate thereof are also encompassed by the invention. Individual dosage forms of the invention are suitable for transdermal, or topical administration

Pharmaceutical compositions and dosage forms used in the invention comprise a compound of formula I, or a pharmaceutically acceptable prodrug, polymorph, salt, solvate, hydrate, or clathrate thereof. Pharmaceutical compositions and dosage forms of the invention typically also comprise one or more pharmaceutically acceptable excipients.

A particular pharmaceutical composition encompassed by this embodiment comprises a compound of formula I, or a pharmaceutically acceptable polymorph, prodrug, salt, solvate, hydrate, or clathrate thereof, and at least one additional therapeutic agent.

Particularly preferred is the method according to the present invention, wherein the composition for topical use is a slow-release formulation.

Nucleoside analogues (NAs) are known as agents in the treatment of hematological malignancies. Their rapid catabolismcell resistance and overdistribution in the body jeopardize nucleoside analogue chemotherapy. NAs are hydrophilic polar molecules with low membrane permeability, and, thus, require specialized nucleoside transporter proteins to enter cells. Therefore, the abundance and tissue distribution of these transporters contributes to cellular specificity and sensitivity to nucleoside analogues. On the other hand, there are no transporter proteins for ribonucleotides and deoxyribonucleotides in the cell membrane, and their negatively charged phosphate groups prevent their diffusion across the membrane.

Accordingly, therapeutic doses of NAs are particularly high and regularly have to be increased, resulting in severe toxicity and narrow therapeutic index. The major challenge is to concentrate the drug at the tumor site, avoiding its distribution to normal tissues. New drug carriers and biomaterials are being developed to overcome some of these obstacles. Diab, R., et al. ((2007). Nucleoside analogue delivery systems in cancer therapy. Expert Opinion on Drug Delivery, 4 (5), 513-531. https://doi.org/10.1517/17425247.4.5.513) highlight NA delivery systems and discusses new technologies that could improve NA cancer therapy. These administration forms can be readily adjusted to the use in skin diseases.

Particularly preferred is the method according to the present invention, wherein the composition for topical use comprises an adhesive patch, such as a transdermal patch. Respective patches are known in the art, such as in

Preferred is the method according to the present invention, wherein the clitocine is administered at concentrations and/or to reach concentrations in the sub-micromolar range, such as, for example, between about 0.1 μM and about 2 μM, preferably between about 0.8 μM and about 1.8 μM, more preferably between about 1 μM and 1.6 μM. See also examples below.

The magnitude of a prophylactic or therapeutic dose of a particular active ingredient of formula I in the acute or chronic management of a disease or condition will vary, however, with the nature and severity of the disease or condition, and the route by which the active ingredient is administered, here topical administration.

The dose, and perhaps the dose frequency, will also vary according to the age, body weight, and response of the individual patient. Suitable dosing regimens can be readily selected by those skilled in the art with due consideration of such factors. In general, the recommended daily dose range for the conditions described herein lie within the range as above per day. In one embodiment, the compound of formula I is given as a single once-a-day dose. In another embodiment, the compound of formula I is given as divided doses throughout a day. More specifically, the daily dose is administered in a single dose or in equally divided doses.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

The phrases “therapeutically effective amount”, “prophylactically effective amount” and “therapeutically or prophylactically effective amount,” as used herein encompass the above-described dosage amounts and dose frequency schedules. Different therapeutically effective amounts may be applicable for different diseases and conditions, as will be readily known by those of ordinary skill in the art. Similarly, amounts sufficient to treat or prevent such diseases, but insufficient to cause, or sufficient to reduce, adverse effects associated with conventional therapies are also encompassed by the above-described dosage amounts and dose frequency schedules.

As mentioned, the treatment with the compound according to formula I is particularly effective, and preferred is the method according to the present invention, wherein the treatment provides a normalized fold induction of translation of more than about 20, preferably of more than about 50, and more preferably of more than about 80, compared to an untreated control. See also examples, below.

Without being limited by theory, the ability of the compounds of formula I to promote readthrough of premature stop codons makes them useful in the treatment or prevention of any skin disease which is caused in whole or in part by a nonsense mutation. Such diseases can occur due to the decreased amount of active protein produced as a result of premature termination of translation. Without being limited by theory, the compounds of formula I allow the translation of mRNA to continue past the nonsense mutation resulting in the production of full length protein.

A powerful aspect of the invention is that the therapeutic activity of compounds of formula I, outside of the nonsense mutation aspect, are not necessarily limited to a specific skin disease, instead are effective at treating or preventing many diseases associated with a nonsense mutation. Further, the methods of the invention include those that are patient specific, that is, a patient can be screened to determine if their disease is associated with a nonsense mutation. If so, they can then be treated with a compound of the invention more efficiently, specifically and effectively.

The compounds of formula I are useful for treating or preventing skin diseases associated with a nonsense mutation. Diseases that can be treated or prevented by compounds of formula I include, but are not limited to, a skin disease selected from a genodermatosis, such as epidermolysis bullosa.

In a particularly preferred embodiment of the method according to the present invention, the premature translation termination is caused by mutation R635X in the gene LAMB3, or a mutation selected from Arg2332X, Arg2541X, Gly2599X, and Arg2685X in the gene COL7A1.

In a preferred embodiment, the invention encompasses a method of treating or preventing a disease ameliorated by modulation of premature translation termination, or ameliorating one or more symptoms associated therewith comprising contacting a cell with an effective amount of a compound of formula I.

Cells encompassed by the present methods include animal cells, and mammalian cells. In one embodiment, the nonsense mutation is a genetic mutation (i.e., the nonsense codon was present in the progenitor DNA).

In certain embodiments, a compound of formula I is administered to a patient, preferably a mammal, more preferably a human, as a preventative measure against a disease associated with premature translation termination.

In a preferred embodiment, it is first determined that the patient is suffering from a disease associated with premature translation termination. In another embodiment, the patient has undergone a screening process to determine the presence of a nonsense mutation comprising the steps of screening a subject, or cells extracted therefrom, by an acceptable nonsense mutation screening assay. In a preferred embodiment, the DNA of the patient can be sequenced or subjected to Southern Blot, polymerase chain reaction (PCR), use of the Short Tandem Repeat (STR), or polymorphic length restriction fragments (RFLP) analysis to determine if a nonsense mutation is present in the DNA of the patient. In one embodiment, it is determined whether the nonsense mutation is a genetic mutation by comparison of progenitor DNA.

Preferred is the method according to the present invention, wherein the treatment is in combination with at least one additional dermatological drug. Specific methods of the invention further comprise the administration of an additional therapeutic agent (i.e., a therapeutic agent other than a compound of formula I). In certain embodiments of the present invention, the compounds of formula I can be used in combination with at least one other therapeutic agent. Therapeutic agents include, but are not limited to non-opioid analgesics; non-steroid anti-inflammatory agents; antiemetics; β-adrenergic blockers; anticonvulsants; antidepressants; Ca²⁺-channel blockers; anticancer agent and mixtures thereof.

In certain embodiments, the compounds of formula I can be administered or formulated in combination with antibiotics. In certain embodiments, the antibiotic is a macrolide (e.g., tobramycin), a cephalosporin (e.g., cephalexin, cephradine, cefuroxime, cefprozil, cefaclor, cefixime or cefadroxil), a clarithromycin (e.g., clarithromycin), an erthromycin (e.g., erythromycin), a penicillin (e.g., penicillin V) or a quinolone (e.g., ofloxacin, ciprofloxacin or norfloxacin). In a preferred embodiment, the antibiotic is active against Pseudomonas aeruginosa. The compounds of formula I and the other therapeutics agent can act additively or, more preferably, synergistically. In a preferred embodiment, a composition comprising a compound of formula I is administered concurrently with the administration of another therapeutic agent, which can be part of the same composition or in a different composition from that comprising the compounds of formula I. In another embodiment, a compound of formula I is administered prior to or subsequent to the administration of another therapeutic agent.

Another aspect of the present invention relates to a method of modulating premature translation termination in a mammalian skin cell, comprising contacting a mammalian skin cell exhibiting premature translation termination with an effective amount of a compound having the structure of formula I:

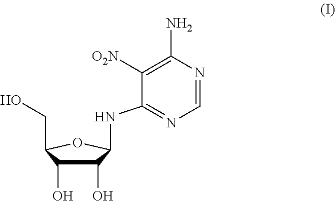

or a pharmaceutically acceptable salt, hydrate, solvate, clathrate, racemate or stereoisomer thereof, wherein the compound, or a pharmaceutically acceptable salt, hydrate, solvate, clathrate or stereoisomer thereof, is administered to the skin cell as a composition for topical use comprising the compound and a pharmaceutically acceptable carrier or diluent.

Apart from the therapeutic aspect, this aspect of the present invention is similar to the aspect as discussed above.

Therefore, as above, preferred is the method according to the present invention, wherein the composition for topical use is selected from a pharmaceutical composition comprising transdermal or topical dosage forms, such as, for example creams, lotions, ointments, gels, solutions, emulsions, suspensions, and combinations thereof.

Preferred is the method according to the present invention, wherein the composition for topical use is a slow release formulation as discussed above.

In this method according to the present invention the clitocine, like above, is also administered at concentrations in the sub-micromolar range, such as, for example, between about 0.1 μM and about 2 μM, preferably between about 0.8 μM and about 1.8 μM, more preferably between about 1 μM and 1.6 μM.

Preferably, in the method according to the present invention the treatment provides a normalized fold induction of translation in the cell of more than about 20, preferably of more than about 50, and more preferably of more than about 80, compared to an untreated control.

In a particularly preferred embodiment of the method according to the present invention, the premature translation termination is caused by mutation R635X in the gene LAMB3, or a mutation selected from Arg2332X, Arg2541X, Gly2599X, and Arg2685X in the gene COL7A1.

In the methods according to the present invention the cell may be a a patient-derived cell or a cell line, I particular a keratinocyte, such as a genetically modified keratinocyte. Thus, cells encompassed by the present methods include animal cells, mammalian cells, bacterial cells, plant cells and virally infected cells. In one embodiment, the nonsense mutation is a genetic mutation (i.e., the nonsense codon was present in the progenitor DNA).

The experiments as provided herein are further of particular relevance for the efficiency of clitocine when administered as a composition for topical use comprising the compound and a pharmaceutically acceptable carrier or diluent, as explained above.

As used herein, “premature translation termination” refers to the result of a mutation that changes a codon corresponding to an amino acid to a stop codon.

As used herein, a “premature termination codon” or “premature stop codon” refers to the occurrence of a stop codon wherein a codon corresponding to an amino acid should be.

As used herein, a “nonsense mutation” is a point mutation changing a codon corresponding to an amino acid to a stop codon. The nonsense mutation can be either a genetic mutation or a somatic mutation. A genetic mutation is one that occurs in a germ cell (i.e., wherein the nonsense codon was present in the progenitor DNA). A somatic mutation is one that occurs in the somatic tissue (i.e., non germline tissue) through mutagenesis and will not be heritable. The somatic mutation may be naturally occurring or the result of exposure to a chemical, radiation, UV rays, X-rays or other environmental factors.

As used herein, “nonsense suppression” refers to the inhibition or suppression of premature translation and/or nonsense-mediated mRNA decay.

As used herein, “modulation of premature translation termination” refers to the regulation of gene expression by altering the level of nonsense suppression. For example, if it is desirable to increase production of a defective protein encoded by a gene with a premature stop codon, i.e., to permit readthrough of the premature stop codon of the disease gene so translation of the gene can occur, then modulation of premature translation termination entails up-regulation of nonsense suppression. Conversely, if it is desirable to promote the degradation of an mRNA with a premature stop codon, then modulation of premature translation termination entails down-regulation of nonsense suppression.

As used herein, the term “disease” means a condition in the patient.

As used herein, the term “patient” means an animal (e.g., cow, horse, sheep, pig, chicken, turkey, quail, cat, dog, mouse, rat, rabbit, guinea pig, etc.), preferably a mammal such as a non-primate and a primate (e.g., monkey and human), most preferably a human. In certain embodiments, the patient is an infant, child, adolescent or adult. In one embodiment, it has been determined through pre-screening that the patient possesses a nonsense mutation. In another embodiment, it has been determined through pre-screening which non-sense mutation the patient has (i.e., UAA, UGA, or UAG). In another embodiment, the patient has a genetic mutation.

Epidermolysis bullosa (EB)-exemplary for this group of diseases-describes a diverse group of rare, blistering genodermatoses. Characterized by extreme skin fragility upon minor mechanical trauma, the most severe forms often result from nonsense mutations that lead to premature translation termination and loss of function of essential proteins at the dermo-epidermal junction

As used herein, a “therapeutically effective amount” refers to that amount of the compound of formula I or other active ingredient sufficient to provide a therapeutic benefit in the treatment or management of the disease or to delay or minimize symptoms associated with the disease. Further, a therapeutically effective amount with respect to a compound of formula I means that amount of therapeutic agent alone, or in combination with other therapies, that provides a therapeutic benefit in the treatment or management of the disease. Used in connection with an amount of a compound of formula I, the term can encompass an amount that improves overall therapy, reduces or avoids symptoms or causes of disease, or enhances the therapeutic efficacy of or synergies with another therapeutic agent.

As used herein, a “prophylactically effective amount” refers to that amount of a compound of formula I or other active ingredient sufficient to result in the prevention, recurrence or spread of the disease. A prophylactically effective amount may refer to the amount sufficient to prevent initial disease, the recurrence or spread of the disease or the occurrence of the disease in a patient, including but not limited to those predisposed to the disease. A prophylactically effective amount may also refer to the amount that provides a prophylactic benefit in the prevention of the disease. Further, a prophylactically effective amount with respect to a compound of formula I means that amount alone, or in combination with other agents, that provides a prophylactic benefit in the prevention of the disease. Used in connection with an amount of a compound of formula I, the term can encompass an amount that improves overall prophylaxis or enhances the prophylactic efficacy of or synergies with another prophylactic agent.

As used herein, “in combination” refers to the use of more than one prophylactic and/or therapeutic agents.

As used herein, the terms “prevent”, “preventing” and “prevention” refer to the prevention of the onset, recurrence or spread of the disease in a subject resulting from the administration of a prophylactic or therapeutic agent.

As used herein, the terms “treat”, “teating” and “teatment” refer to the eradication or amelioration of the disease or symptoms associated with the disease. In certain embodiments, such terms refer to minimizing the spread or worsening of the disease resulting from the administration of one or more prophylactic or therapeutic agents to a subject with such a disease.

Another aspect of the present invention relates to a method for manufacturing a pharmaceutical composition for treating or preventing a nonsense-mediated disease as described herein, comprising the steps of formulating the compound of formula I into a pharmaceutical composition for topical administration.

Carriers, excipients and strategies to formulate a pharmaceutical composition to be administered topically, e.g. in the form of lotions, gels, ointments or creams, or in nasal or a suppository or patch form are well known to the person of skill and described in the respective literature.

In the context of the present invention, the term “about” shall mean a deviation of +/−10% from a given value, unless indicated otherwise.

Clitocine is a potent inducer of SCR in the EB-disease context. Clitocine induces significantly higher levels of SCR compared to G418 or gentamicin. At the same time, clitocine is better tolerated than G418 or gentamicin as indicated by milder effects on cell viability.

Therefore, the results show that clitocine is particularly suitable for a topical treatment for cases of genodermatoses, such as EB, caused by nonsense variants.

The present invention relates to the following items:
Item 1: A method of treating a skin disease responsive to modulation of premature translation termination, comprising administering to a mammalian patient in need thereof an effective amount of a compound having the structure of formula I:

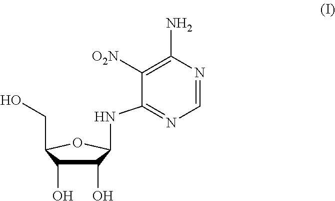

or a pharmaceutically acceptable salt, hydrate, solvate, clathrate, racemate or stereoisomer thereof, wherein the compound, or a pharmaceutically acceptable salt, hydrate, solvate, clathrate or stereoisomer thereof, is administered as a composition for topical use comprising the compound and a pharmaceutically acceptable carrier or diluent.
Item 2. The method according to Item 1, wherein the composition for topical use is selected from a pharmaceutical composition comprising transdermal or topical dosage forms, such as, for example creams, lotions, ointments, gels, solutions, emulsions, suspensions, and combinations thereof.
Item 3. The method according to Item 1, wherein the composition for topical use is a slow release formulation.
Item 4. The method according to Item 1, wherein the composition for topical use comprises a adhesive patch, such as a transdermal patch.
Item 5. The method according to Item 1, wherein the clitocine is administered at concentrations in the sub-micromolar range, such as, for example, between about 0.1 μM and about 2 μM, preferably between about 0.8 UM and about 1.8 μM, more preferably between about 1 μM and 1.6 μM.
Item 6. The method according to Item 1, wherein the treatment provides a normalized fold induction of translation of more than about 20, preferably of more than about 50, and more preferably of more than about 80, compared to an untreated control.
Item 7. The method according to Item 1, wherein the skin disease is selected from a genodermatosis, such as epidermolysis bullosa.
Item 8. The method according to Item 1, wherein the premature translation termination is caused by mutation R635X in the gene LAMB3, or a mutation selected from Arg2332X, Arg2541X, Gly2599X, and Arg2685X in the gene COL7A1.
Item 9. The method according to Item 1, wherein the treatment is in combination with at least one additional dermatological drug.
Item 10. A method of modulating premature translation termination in a mammalian skin cell, comprising contacting a mammalian skin cell exhibiting premature translation termination with an effective amount of a compound having the structure of formula I:

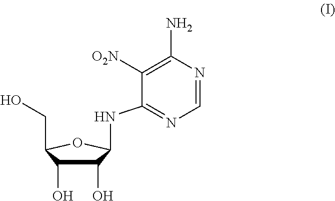

or a pharmaceutically acceptable salt, hydrate, solvate, clathrate, racemate or stereoisomer thereof, wherein the compound, or a pharmaceutically acceptable salt, hydrate, solvate, clathrate or stereoisomer thereof, is administered to the skin cell as a composition for topical use comprising the compound and a pharmaceutically acceptable carrier or diluent.
Item 11. The method according to Item 10, wherein the composition for topical use is selected from a pharmaceutical composition comprising transdermal or topical dosage forms, such as, for example creams, lotions, ointments, gels, solutions, emulsions, suspensions, and combinations thereof.
Item 12. The method according to Item 10, wherein the composition for topical use is a slow release formulation.
Item 13. The method according to Item 10, wherein the clitocine is administered at concentrations in the sub-micromolar range, such as, for example, between about 0.1 μM and about 2 μM, preferably between about 0.8 μM and about 1.8 μM, more preferably between about 1 μM and 1.6 μM.
Item 14. The method according to Item 10, wherein the treatment provides a normalized fold induction of translation in the cell of more than about 20, preferably of more than about 50, and more preferably of more than about 80, compared to an untreated control.
Item 15. The method according to Item 10, wherein the premature translation termination is caused by mutation R635X in the gene LAMB3, or a mutation selected from Arg2332X, Arg2541X, Gly2599X, and Arg2685X in the gene COL7A1.
Item 16. The method according to Item 10, wherein the cell is a patient-derived cell or a cell line, in particular a keratinocyte, such as a genetically modified keratinocyte.

### BRIEF DESCRIPTION OF THE FIGURES

The invention will now be described further in the following examples with reference to the accompanying figures, nevertheless, without being limited thereto. For the purposes of the present invention, all references as cited are incorporated by reference in their entireties. Citation of any reference of this application is not an admission that the reference is prior art to the application.

FIG. 1 shows that nucleoside analog clitocine induces SCR of a JEB hot spot mutation (NM_000228.3: c.1903C>T; NP_000219.2:p.Arg635*) in stably transduced N/TERT keratinocytes. Cell were treated with the indicated concentrations of clitocine for 24 hours. Treatment with G418 (0.5 mg/ml) or DMSO served as positive or negative control, respectively. Renilla luciferase activity (R, normalization control) and firefly luciferase activity (F, indicative of SCR) were quantified in relative light units (RLUs). Normalized fold changes (F/R ratio normalized to DMSO) are indicated. * p<0.05 ** p<0.005 *** p<0.0005.

FIG. 2 shows dose-response experiment that reveal clitocine concentration for optimal induction SCR. Stably transduced N/TERT keratinocytes (as described in FIG. 1) were treated with the indicated concentrations of clitocine for 24 hours. * p<0.05 ** p<0.005 *** p<0.0005.

FIG. 3 shows that clitocine induces SCR of a JEB hot spot mutation (NM 000228.3: c.1903C>T; NP_000219.2:p.Arg635*) in stably transduced HEK293AD cells. Cells were treated with the indicated concentrations of clitocine (0.5 μM), G418 (0.5 mg/ml), or gentamicin (1 and 2 mg/ml) for 24 hours. DMSO served as negative control. Renilla luciferase activity (R, normalization control) and firefly luciferase activity (F, indicative of SCR) were quantified in relative light units (RLUs). Normalized fold changes (F/R ratio normalized to DMSO) are indicated. * p<0.05 ** p<0.005 *** p<0.0005.

FIG. 4 shows that clitocine induces SCR of four different COL7A1 variants (p. Arg2332*, p.Arg2541*, p.Gly2599*, and p.Arg2685*) in transiently transfected HEK293AD cells. Cells were treated with the indicated concentrations of clitocine for 24 hours (n≥3 per experimental group). Firefly luciferase activity (Fluc, indicative of SCR) was quantified in relative light units (RLUs) using the Dual-Glo Luciferase Assay System. * p<0.05 ** p<0.01 *** p<0.001 **** p<0.0001.

FIG. 5 shows that clitocine is well tolerated in RDEB patient cells. RDEB662 cells were treated with indicated concentrations of clitocine, G418 (0.5 mg/ml), or gentamicin (2 mg/ml) for 24 (A), 48 (B), or 72 hours (C). DMSO served as negative control. Percent viability normalized to vehicle control (Opti-MEM) was determined by CellTiter Glo® Luminescent Cell Viability Assay. *p<0.05 ** p<0.005 *** p<0.0005.

### EXAMPLES

### The Non-Aminoglycoside Compound Clitocine is a Potent Inducer of Stop Codon Read-Through in Model Systems of Epidermolysis Bullosa

The inventors assessed the efficacy of the nucleoside analog clitocine to induce stop codon readthrough (SCR) in different epidermolysis bullosa (EB) disease contexts. Recurrent nonsense variants in junctional (JEB) and recessive dystrophic EB (RDEB) were tested.

The experiments as provided herein are further of particular relevance for the efficiency of clitocine when administered as a composition for topical use comprising the compound and a pharmaceutically acceptable carrier or diluent.

### Methods

The inventors adapted a dual-luciferase reporter system to evaluate SCR in the context of JEB and RDEB at high capacity. Under the control of a CMV promoter, Renilla (as normalization control) and firefly luciferase (for measurement of SCR) were cloned in tandem, separated by a nonsense codon and the surrounding sequence context (3 codons up- and downstream of the respective nonsense variant).

The following nonsense variants and their sequence context were selected: the LAMB3 hotspot mutation p.Arg635* for JEB and the COL7A1 variants p. Arg2332*, p.Arg2541*, p.Gly2599*, and p.Arg2685* for RDEB. Initially, reporter constructs were transiently transfected into HEK293T cells and experimentally validated with positive (G418) and negative (DMSO) controls. Aiming for the highest disease relevance, the inventors subsequently integrated the reporter constructs in the N/TERT keratinocytes cell line by lentiviral transduction, allowing controlled and standardized experimentation. Firefly and Renilla luciferase activity were quantified in relative light units (RLUs) using the Dual-Glo® Luciferase Assay System. Effects of clitocine (and controls) on cell viability were assessed by time- and dose-response experiments in a patient-derived RDEB cell line (RDEB662) using the CellTiter Glo® Luminescent Cell Viability Assay.

### Results

The magnitude of induced SCR depends on numerous factors, some of which are highly disease-specific. Accordingly, the efficacy of a given compound to induce SCR varies for specific sequence contexts of pathogenic nonsense variants or different cell types and tissues, and is also dependent from the mode of administration, in particular topical administration.

The investors tested the efficacy of clitocine to induce SCR for different nonsense variants associated with JEB and RDEB in keratinocytes.

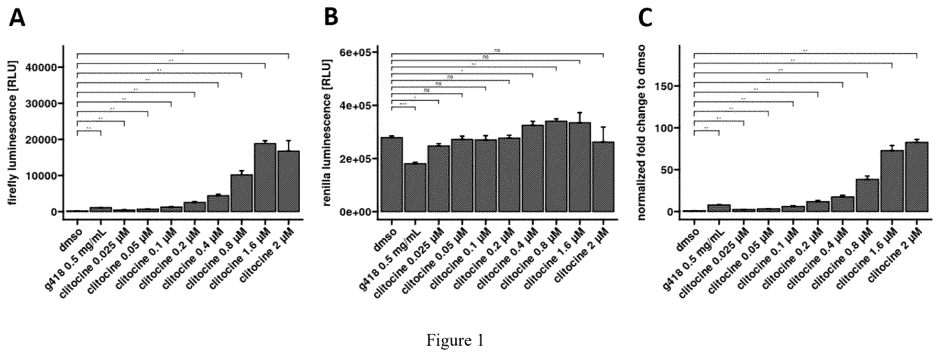

Clitocine induced SCR of the LAMB3 hotspot mutation R635X (NM_000228.3: c. 1903C>T; NP_000219.2:p.Arg635Ter) at concentrations in the sub-micromolar range. High levels of R635X SCR were achieved, as indicated by prominent firefly luciferase activity in a dual luciferase reporter assay. Specifically, treatment with G418 (0.5 mg/mL) resulted in a normalized fold induction of 10 compared to DMSO control. At the same time, the inventors recorded a normalized fold induction of 80 upon treatment with 2 μM clitocine (FIG. 1). In this assay, RLuc signals are used for normalization, while FLuc signals indicate SCR. Normalized readthrough is calculated as the ratio of FLuc/RLuc signals.

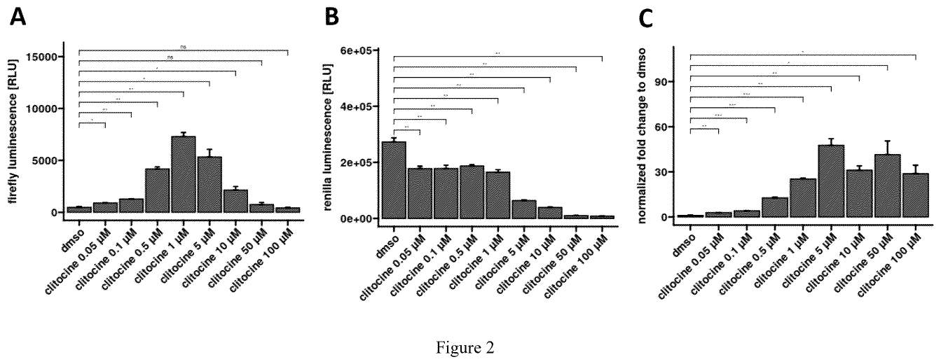

Clitocine induced SCR in a dose-dependent manner. A slight drop in RLuc signals at 2 μM indicated a general effect on translation or toxicity. This effect was more pronounced at higher concentrations, such as would be achieved in a topical administration, where treatment with clitocine at concentrations of 5 μM or higher led to a marked drop-off of RLuc signals (FIG. 2).

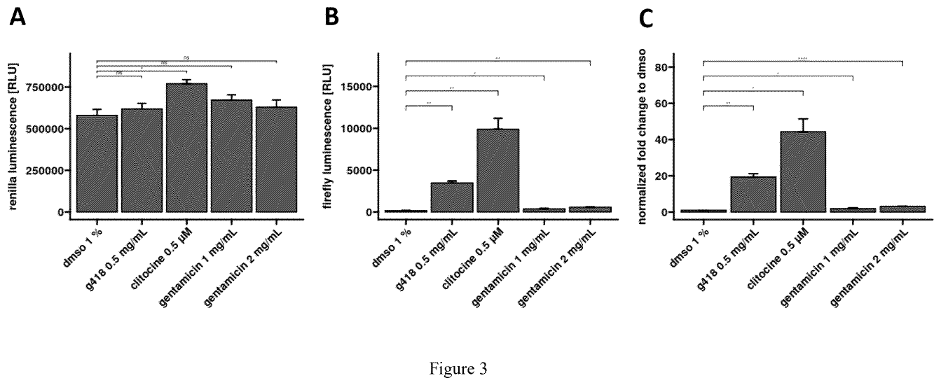

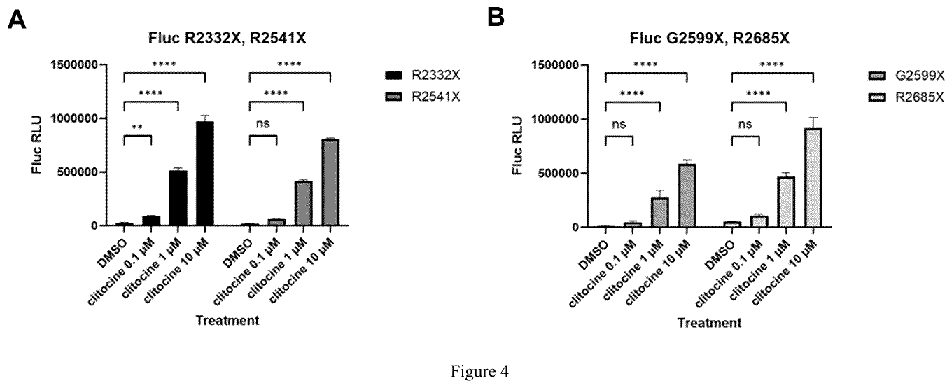

The inventors also compared the efficacy of clitocine and gentamicin (already in clinical trials in EB) to induce SCR of R635X. As shown in FIG. 3, SCR induced by clitocine was much higher than SCR induced by gentamicin (or G418). Clitocine also was efficacious for three different RDEB-related nonsense variants in COL7A1. Clitocine induced SCR for p. Arg2332*, p.Arg2541*, p.Gly2599*, and p.Arg2685* at comparable levels (FIG. 4).

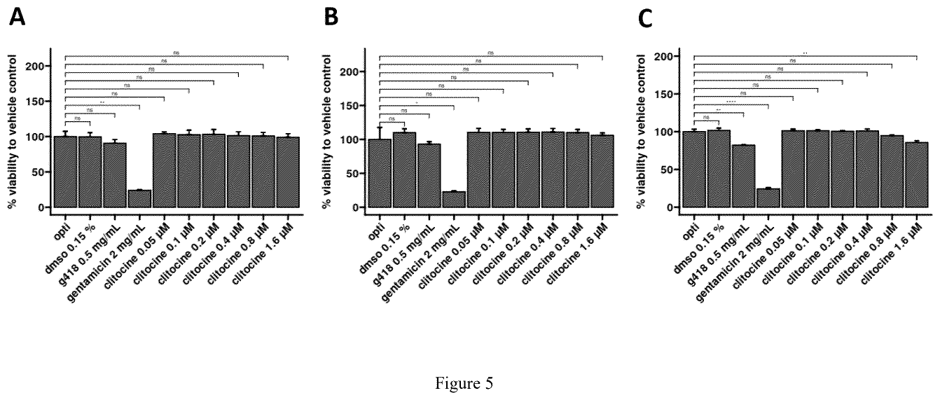

Finally, the inventors also assessed the effects of clitocine treatment on cell viability in patient-derived cells. Clitocine was well tolerated at concentrations relevant to SCR (FIG. 5). Only minimal effects on cell viability were observed at concentrations of 1.6 μM after 72 hours of treatment. In comparison, treatment with G418 or gentamicin had pronounced effects on cell viability.

### CONCLUSION

Clitocine is a potent inducer of SCR in the EB-disease context. Clitocine induces significantly higher levels of SCR compared to G418 or gentamicin. At the same time, clitocine is better tolerated than G418 or gentamicin as indicated by milder effects on cell viability.

Therefore, the results show that clitocine is particularly suitable for a topical treatment for cases of genodermatoses, such as EB, caused by nonsense variants.

1. A method of treating a skin disease responsive to modulation of premature translation termination, comprising administering to a mammalian patient in need thereof an effective amount of a compound having the structure of formula I: 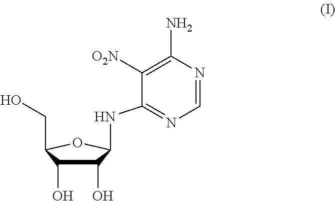
or a pharmaceutically acceptable salt, hydrate, solvate, clathrate, racemate or stereoisomer thereof, wherein the compound, or a pharmaceutically acceptable salt, hydrate, solvate, clathrate or stereoisomer thereof, is administered as a composition for topical use comprising the compound and a pharmaceutically acceptable carrier or diluent.

2. The method according to claim 1, wherein the composition for topical use is a pharmaceutical composition comprising a transdermal or topical dosage form selected from creams, lotions, ointments, gels, solutions, emulsions, suspensions, and combinations thereof.

3. The method according to claim 1, wherein the composition for topical use is a slow release formulation.

4. The method according to claim 1, wherein the composition for topical use comprises an adhesive patch.

5. The method according to claim 1, wherein the clitocine is administered at a concentration of between about 0.1 μM and about 2 μM.

6. The method according to claim 1, wherein the treatment provides a normalized fold induction of translation of more than about 20 compared to an untreated control.

7. The method according to claim 1, wherein the skin disease is selected from a genodermatosis, such as epidermolysis bullosa.

8. The method according to claim 1, wherein the premature translation termination is caused by mutation R635X in the gene LAMB3, or a mutation selected from Arg2332X, Arg2541X, Gly2599X, and Arg2685X in the gene COL7A1.

9. The method according to claim 1, wherein the treatment is in combination with at least one additional dermatological drug.

10. A method of modulating premature translation termination in a mammalian skin cell, comprising contacting a mammalian skin cell exhibiting premature translation termination with an effective amount of a compound having the structure of formula I: 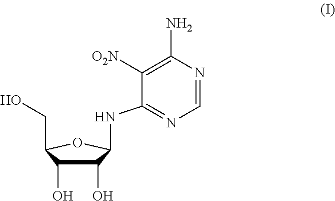
or a pharmaceutically acceptable salt, hydrate, solvate, clathrate, racemate or stereoisomer thereof, wherein the compound, or a pharmaceutically acceptable salt, hydrate, solvate, clathrate or stereoisomer thereof, is administered to the skin cell as a composition for topical use comprising the compound and a pharmaceutically acceptable carrier or diluent.

11. The method according to claim 10, wherein the composition for topical use is a pharmaceutical composition comprising a transdermal or topical dosage form selected from creams, lotions, ointments, gels, solutions, emulsions, suspensions, and combinations thereof.

12. The method according to claim 10, wherein the composition for topical use is a slow release formulation.

13. The method according to claim 10, wherein the clitocine is administered at a concentration between about 0.1 μM and about 2 μM.

14. The method according to claim 10, wherein the treatment provides a normalized fold induction of translation in the cell of more than about 20 compared to an untreated control.

15. The method according to claim 10, wherein the premature translation termination is caused by mutation R635X in the gene LAMB3, or a mutation selected from Arg2332X, Arg2541X, Gly2599X, and Arg2685X in the gene COL7A1.

16. The method according to claim 10, wherein the cell is a keratinocyte.

17. The method according to claim 1, wherein said administration is done via a transdermal patch.

18. The method according to claim 7, wherein the skin disease is epidermolysis bullosa.
